(19) United States
(12) Patent Application Publication
(10) Pub. No.: US 2025/0032031 A1
(43) Pub. Date: Jan. 30, 2025

(54) CONDUCTED PREMATURE ATRIAL CONTRACTION (PAC) BURDEN ESTIMATION ALGORITHM IN ICM

(71) Applicant: Cardiac Pacemakers, Inc., St. Paul, MN (US)

(72) Inventors: Abhijit Rajan, Shoreview, MN (US); David L. Perschbacher, Blaine, MN (US); Krzysztof Z. Siejko, Maple Grove, MN (US); Vasanth Ravikumar, Minneapolis, MN (US)

(21) Appl. No.: 18/780,027

(22) Filed: Jul. 22, 2024

Related U.S. Application Data
(60) Provisional application No. 63/528,563, filed on Jul. 24, 2023.

(51) Int. Cl. A61B 5/349; A61B 5/00; A61B 5/36; A61N 1/365; A61N 1/39

(52) U.S. Cl. CPC A61B 5/349; A61B 5/36; A61B 5/746; A61N 1/36592; A61N 1/3987

(57) ABSTRACT

Systems and methods are disclosed to an ambulatory medical device comprising a cardiac signal sensing circuit configured to sense a cardiac signal representative of cardiac activity of a patient when connected to electrodes, and a control circuit. The control circuit is configured to monitor cardiac depolarizations in the sensed cardiac signal, detect a bimodal distribution of heart rate of the patient, identify cardiac depolarization intervals shorter than a predetermined interval threshold, identify premature atrial contractions (PACs) in the sensed cardiac signal that are conducted normally and conducted aberrantly, and count a number of conducted PACs and produce an alert related to PC burden of the patient based on the number.

### CLAIM OF PRIORITY

This application claims the benefit of U.S. Provisional Application No. 63/528,563, filed on Jul. 24, 2023, which is hereby incorporated by reference in its entirety.

### TECHNICAL FIELD

This document relates generally to medical devices and more particularly to systems, methods, and devices to estimate conducted premature atrial contraction burden of a patient.

### BACKGROUND

Ambulatory medical devices (AMDs), including implantable, subcutaneous, insertable, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to detect conducted premature atrial contractions (PACs). PACs may be a predictor of Atrial Fibrillation (AF), stroke, and mortality in general. A device determined value of PAC burden can be provided to a user, or a process, based on detection of conducted PACs.

Example 1 includes subject matter (such as an ambulatory medical device (AMD) comprising a cardiac signal sensing circuit configured to sense a cardiac signal representative of cardiac activity of a patient when connected to electrodes, and a control circuit. The control circuit is configured to monitor cardiac depolarizations in the sensed cardiac signal, detect a bimodal distribution of heart rate of the patient, identify cardiac depolarization intervals shorter than a predetermined interval threshold, identify premature atrial contractions (PACs) in the sensed cardiac signal that are conducted normally and conducted aberrantly, and count a number of conducted PACs and produce an alert related to PAC burden of the patient based on the number.

In Example 2, the subject matter of Example 1 optionally includes a control circuit configured to identify the PACs by identifying ectopic P-waves before conducted R-waves in the sensed cardiac signal.

In Example 3, the subject matter of Example 2 optionally includes a control circuit configured to compute correlation between QRS complexes in the sensed cardiac signal, and identify the PACs in the sensed cardiac signal using the correlation.

In Example 4, the subject matter of Example 2 optionally includes a control circuit configured to analyze ectopic P-wave information and morphology of the sensed cardiac signals to distinguish the PACs from premature ventricular contractions (PVCs).

In Example 5, the subject matter of one or any combination of Examples 1-4 optionally includes a control circuit configured to analyze morphology of the sensed cardiac signals to identify the PACs that are normally conducted and the PACs that are aberrantly conducted.

In Example 6, the subject matter of one or any combination of Examples 1-5 optionally includes a control circuit configured to produce a histogram of depolarization intervals of the patient and determine when the histogram is bimodal.

In Example 7, the subject matter of one or nay combination of Examples 1-6 optionally includes a control circuit configured to monitor depolarization intervals of the patient, produce a histogram of the depolarization intervals, detect when the histogram is bimodal, and enable the identifying of the PACs during a predetermined timing window of the sensed cardiac signal.

In Example 8, the subject matter of Example 7 optionally includes a communication circuit configured to communicate information wirelessly to a separate device; and a control circuit configured to monitor the depolarization intervals over multiple days, communicate an alert to the separate device when determining that the histogram is bimodal, and receive a command from the separate device to enable the identifying of the PACs in response to a command received from the separate device.

In Example 9, the subject matter of one or any combination of Examples 1-7 optionally includes a communication circuit operatively configured to communicate information wirelessly to a separate device; and a control circuit configured to produce a value of conducted PAC burden and include the value of conducted PAC burden in the alert related to PAC burden of the patient.

Example 10 includes subject matter (such as a method of operating an AMD) or can optionally be combined with one or any combination of Examples 1-9 to include such subject matter, comprising monitoring cardiac depolarizations of a patient in a sensed cardiac signal sensed using the AMD, detecting a bimodal distribution of heart rate of the patient, identifying cardiac depolarization intervals shorter than a predetermined interval threshold, identifying premature atrial contractions (PACs) in the sensed cardiac signal that are conducted normally and conducted aberrantly, and determining a number of conducted PACs and producing an alert related to PAC burden of the patient based on the number.

In Example 11, the subject matter of Example 10 optionally includes the AMD detecting ectopic P-waves before a conducted R-wave in the sensed cardiac signal.

In Example 12, the subject matter of Example 11 optionally includes the AMD using ectopic P-wave information and morphology of the sensed cardiac signals to distinguish PACs from premature ventricular contractions (PVCs).

In Example 13, the subject matter of one or any combination of Examples 10-12 optionally includes the AMD determining when the identified PACs are normally conducted and aberrantly conducted using analysis of morphology of the sensed depolarization signal.

In Example 14, the subject matter of one or any combination of Examples 10-13 optionally includes the AMD determining when the identified PACs are normally conducted and aberrantly conducted using analysis of the morphology of the sensed depolarization signal.

In Example 15, the subject matter of one or any combination of Examples 10-14 optionally includes monitoring the depolarization intervals of the patient, producing a histogram of the depolarization intervals, determining that the histogram is bimodal, and enabling sensing of the depolarization signal in response to the determining that the histogram is bimodal and enabling the identifying of the conducted PACs during a predetermined timing window of the sensed depolarization signal.

In Example 16, the subject matter of Example 15 optionally includes communicating an alert to a separate device when determining that the histogram is bimodal; and enabling the sensing of the depolarization signal and the identifying of the conducted PACs in response to a command received from the separate device.

In Example 17, the subject matter of one or any combination of Examples 10-16 optionally includes the AMD computing a value of PAC burden using the determined number of conducted PACs.

Example 18 includes subject matter (such as a cardiac rhythm management (CRM) system) or can optionally be combined with one or any combination of Examples 1-17 to include such subject matter, comprising an implantable AMD and an external device. The external device includes a communication circuit configured to communicate information wirelessly with the AMD when implanted. The AMD is includes a cardiac signal sensing circuit configured to sense a cardiac signal representative of cardiac activity of a patient when connected to electrodes, a communication circuit configured to wirelessly communicate information to a separate device, and a control circuit. The control circuit is configured to monitor cardiac depolarizations in the sensed cardiac signal, detect a bimodal distribution of heart rate of the patient, identify cardiac depolarization intervals shorter than a predetermined interval threshold in response to detecting the bimodal heart rate, identify conducted R-waves in the sensed cardiac signal and detect ectopic P-waves prior to the conducted R-waves, count a number of conducted ectopic P-waves, and produce an alert related to PAC burden of the patient based on the number of conducted ectopic P-waves and communicate the alert to the external device.

In Example 19, the subject matter of Example 18 optionally includes the external device configured to retrieve information stored in memory of the AMD, receive the alert related to PAC burden in the retrieved information, and display the alert related to PAC burden to a user of the external device.

In Example 20, the subject matter of one or both of Examples 18 and 19 optionally includes the AMD including a therapy circuit to provide electrical pacing therapy to the patient when coupled to the electrodes; and the external device being an AMD programmer configured to communicate a command to the AMD to change a therapy mode in response to receiving the alert related to PAC burden.

The Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a patient management system.

FIG. 2 illustrates an example of an ambulatory medical device (IMD) electrically coupled to a heart.

FIG. 3 is a block diagram of the electronic circuits of an AMD.

FIG. 4 is a block diagram of an external device that communicates information with an AMD.

FIGS. 5-7 are illustrations of examples of strip chart recordings of cardiac depolarizations.

FIG. 8 is a flow diagram of a method of operating a CRM system.

FIG. 9 is an illustration of a histogram of heart rate.

### DETAILED DESCRIPTION

Ambulatory medical devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

A sinus node located in the atria typically initiates cardiac contractions of a patient by regularly occurring sinus impulses that cause atrial depolarization. A premature atrial contraction (PAC) can occur in a patient when an ectopic focus in the atria discharges to start an atrial depolarization before a sinus impulse. PACs may be a predictor of Atrial Fibrillation (AF), stroke, and mortality in general. The present inventors have recognized, among other things, systems, and methods to estimate PAC burden can be useful to a physician in monitoring the health of a heart failure patient.

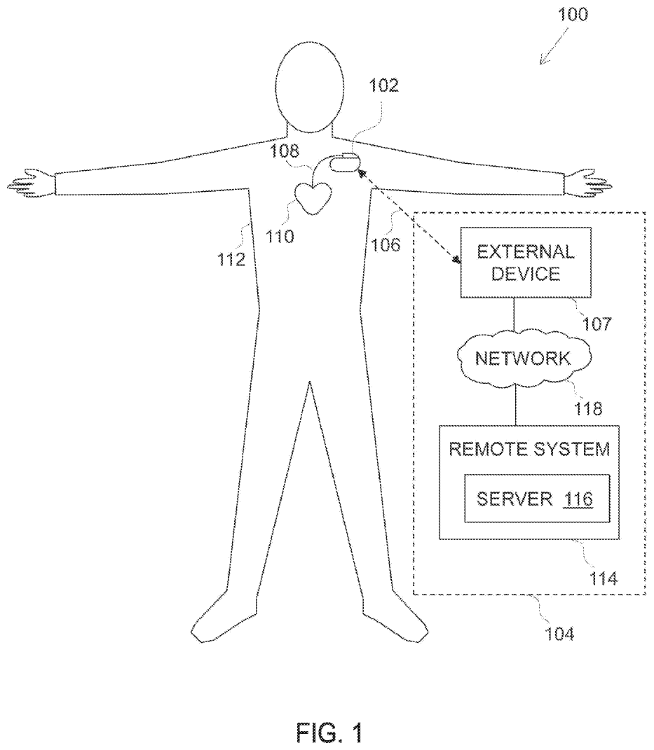

FIG. 1 illustrates portions of an example of a Cardiac Rhythm Management (CRM) system 100 and portions of an environment in which the CRM system 100 can be used. The CRM system 100 can include an AMD 102 that is implantable, an external system 104, and a communication link such as a telemetry link 106. The AMD 102 can include an electronic unit coupled by a cardiac lead 108, or additional leads, to a heart 110 of a subject 112. Examples of the AMD 102 can include, but are not limited to, pacemakers, pacemaker/defibrillators, cardiac resynchronization devices, cardiac remodeling control devices, and insertable cardiac monitors (ICMs). In an example, the AMD 102 can be configured to monitor health of the heart 110 and determine one or more abnormalities associated with the heart 110. The AMD 102 can take a necessary action, such as stimulating one or more portions of the heart 110 through the lead 108, to treat the one or more abnormalities.

In an example, the external system 104 can include an external device 107 configured to communicate bi-directionally with the AMD 102 such as through the telemetry link 106. For example, the external device 107 can include a programmer to program the AMD 102 to provide one or more therapies to the heart 110. In an example, the external device 107 can program the AMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the AMD 102 through the telemetry link 106. Examples of such transmitted data can include programming instructions for the AMD 102 to acquire physiological data, perform at least one self-diagnostic test (such as for a device operational status), or deliver at least one therapy or any other data. In an example, the AMD 102 can be configured to transmit data to the external device 107 through the telemetry link 106. This transmitted data can include real-time physiological data acquired by the AMD 102 or stored in the AMD 102, therapy history data, an operational status of the AMD 102 (e.g., battery status or lead impedance), and the like. The telemetry link 106 can include an inductive telemetry link or a far-field radio-frequency telemetry link.

In an example, the external device 107 can be a part of a CRM system 100 that can include other devices such as a remote system 114 for remotely programming the AMD 102. In an example, the remote system 114 can be configured to include a server 116 that can communicate with the external device 107 through a telecommunication network 118 such as to access the AMD 102 to remotely monitor the health of the heart 110 or adjust parameters associated with the one or more therapies.

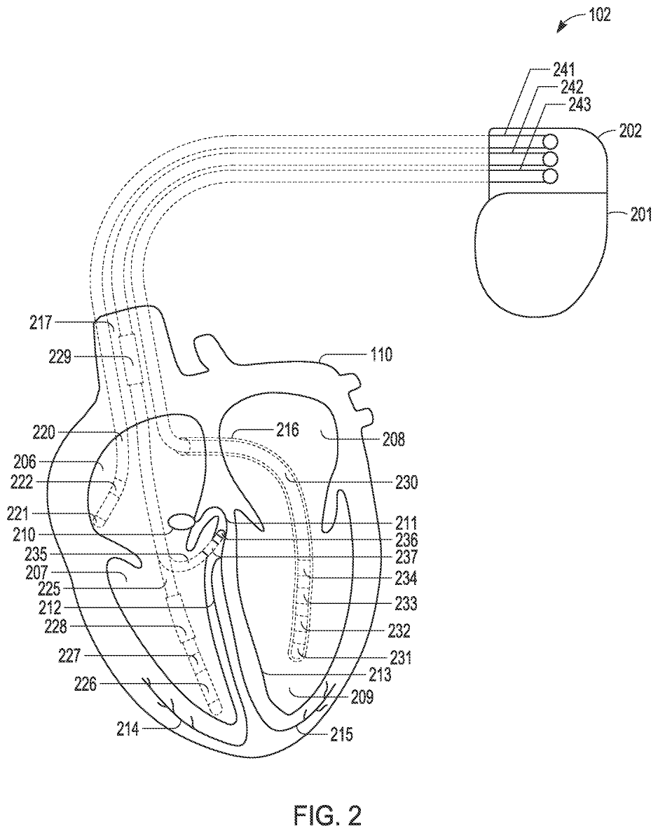

FIG. 2 illustrates an AMD 102 that is an implantable medical device (IMD). The AMD 102 is electrically coupled to a heart 110, such as through one or more leads coupled to the AMD 102 through one or more lead ports, such as first, second, or third lead ports 241, 242, 243 in a header 202 of the AMD 102. In an example, the AMD 102 can include an antenna, such as in the header 202, configured to enable communication with an external system and one or more electronic circuits in a hermetically sealed housing (CAN) 201. The AMD 102 illustrates an example medical device (or a medical device system) as described herein.

The AMD 102 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the AMD 102 can include one or more electrodes or other sensors (e.g., a pressure sensor, an accelerometer, a gyroscope, a microphone, etc.) powered by a power source in the AMD 102. The one or more electrodes or other sensors of the leads, the AMD 102, or a combination thereof, can be configured detect physiologic information from, or provide one or more therapies or stimulation to, the patient.

The AMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

In an example, the AMD 102 can sense impedance such as between electrodes located on one or more of the leads or the CAN 201. The AMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the AMD 102 can be configured to inject current between an electrode on one or more of the first, second, third, or fourth leads 220, 225, 230, 235 and the CAN 201, and to sense the resultant voltage between the same or different electrodes and the CAN 201.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

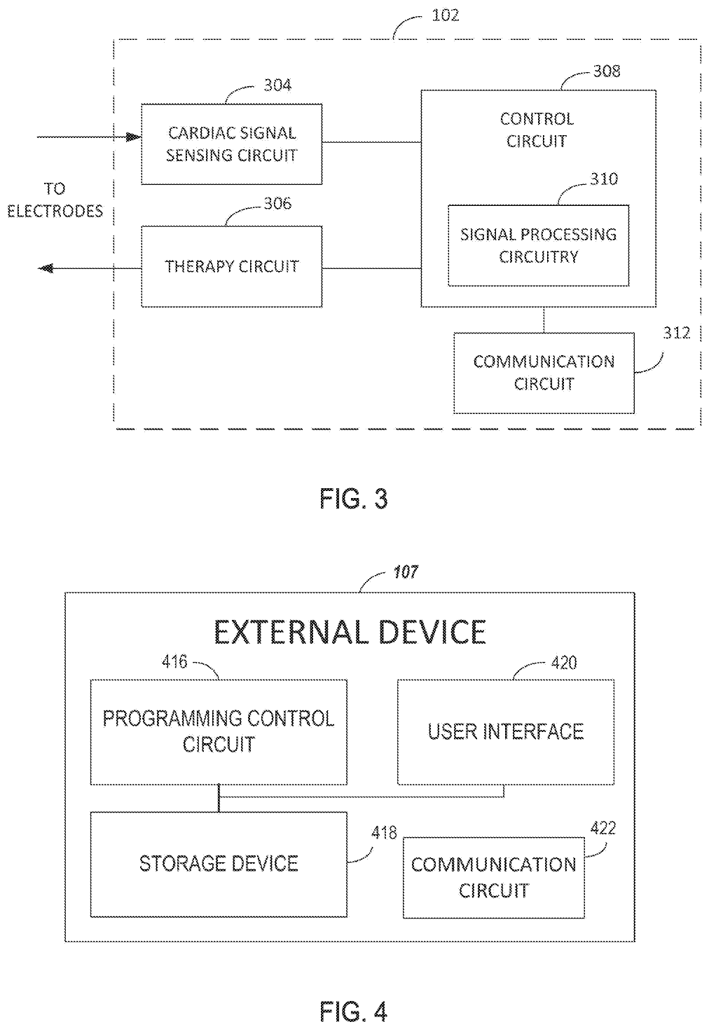

FIG. 3 is a block diagram of portions of electronic circuits of an AMD 102 that is implantable. The AMD 102 can be coupled to multiple implantable electrodes, such as the electrode arrangement described in the example of FIG. 2. The AMD 102 includes a cardiac signal sensing circuit 304, a therapy circuit 306, a communication circuit 312, and a control circuit 308. The therapy circuit 306 provides electrical pacing stimulation energy to the heart of the patient when operatively connected to pacing electrodes of the system. The pacing electrodes can include any of the pacing electrodes in FIG. 2, such as electrodes configured placement in or near the RA, RV, LV, His Bundle, or left bundle branches, and an electrode of the CAN 201.

The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by signal processing circuitry 310 of the control circuit 308. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc. Far-field cardiac signals can be sensed using the electrode of the CAN. In some examples, the AMD 102 is a diagnostic-only device and does not include a therapy circuit 306.

The control circuit 308 may include a digital signal processor, application specific integrated circuit (ASIC), field programmable gate array (FPGA), microprocessor, or other type of processor, interpreting or executing instructions in software or firmware. In some examples, the control circuit 308 may include a state machine or sequencer that is implemented in hardware circuits. The control circuit 308 may include any combination of hardware, firmware, or software. The control circuit 308 includes electronic circuitry (e.g., signal processing circuitry 310) to perform the functions described herein. A circuit may include software, hardware, firmware or any combination thereof. For example, the circuit may include instructions in software executing on the control circuit 308. Multiple functions may be performed by one or more circuits of the control circuit 308.

The control circuit 308 uses the communication circuit 312 to communicate information wirelessly with a separate device. FIG. 4 is a block diagram of portions of an example of an external device 107 (e.g., external device 107 of the CRM system 100 in FIG. 1) to communicate with the AMD 102 of FIG. 3. The external device 107 may be a programming device for the AMD 102. Programming device includes a storage device 418, a programming control circuit 416, a user interface 420, and a communication circuit 422. Programming control circuit 416 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or portions thereof, and a programmable logic circuit or a portion thereof. The storage device 418 may be a memory integral to the programming control circuit 416, or a separate memory device. Communication circuit 422 communicates information wirelessly with the AMD 102 using near-field inductive wireless signals or far-field radio-frequency signals. The programming device can be used to program pacing therapy parameters and other information in the AMD 102.

Some heart failure patients experience PACs. Because PACs can be a predictor of AF, stroke, and mortality, knowledge of the number of P-waves or percentage of P-waves that are PACs (PAC burden) for a heart failure patient can be useful to physicians for effective titration of drug therapy, or other therapy.

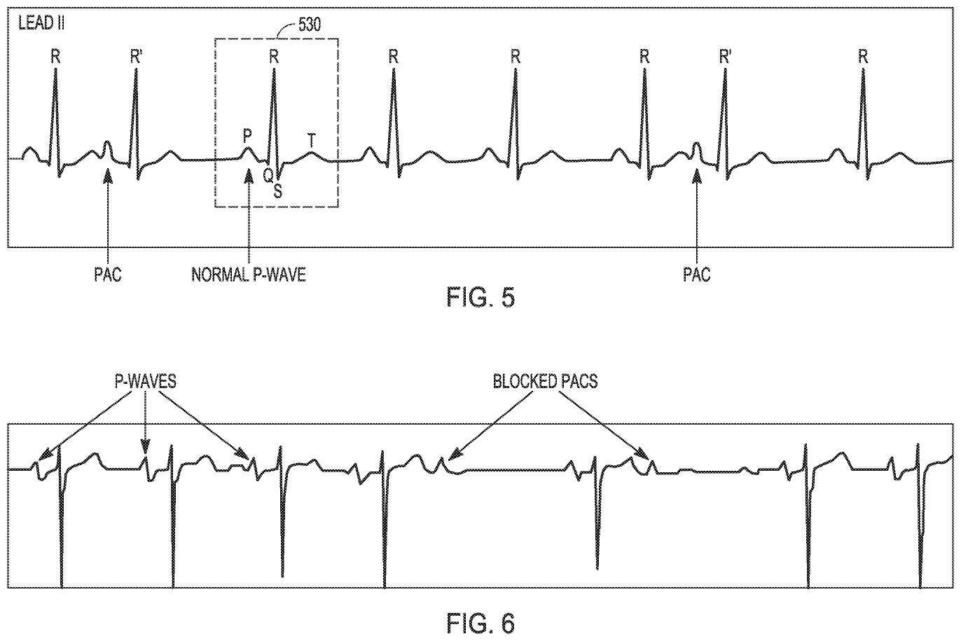

FIG. 5 is an illustration of an example of a strip chart recording of cardiac depolarizations. Depolarization 530 shows the P-wave, Q-wave, R-wave, S-wave, and T-wave of the PQRST complex of the depolarization 530. The P-wave of the depolarization is a normal P-wave representative of contraction of the atria in response to an impulse from the sinus node. FIG. 5 also shows two PACs. The PACs occur earlier relative to the previous QRS complex than a normal P-wave. In the example of FIG. 5, the PACs are conducted through the atrioventricular (AV) node 210 to the ventricles. The R-waves resulting from the PACs are labeled R′.

FIG. 6 is an illustration of another example of a strip chart recording of cardiac depolarizations. The example of FIG. 6 shows normal P-waves and two PACs that are blocked and not conducted through the AV node 210. PACS can be normally conducted, non-conducted or blocked, and aberrantly or abnormally conducted.

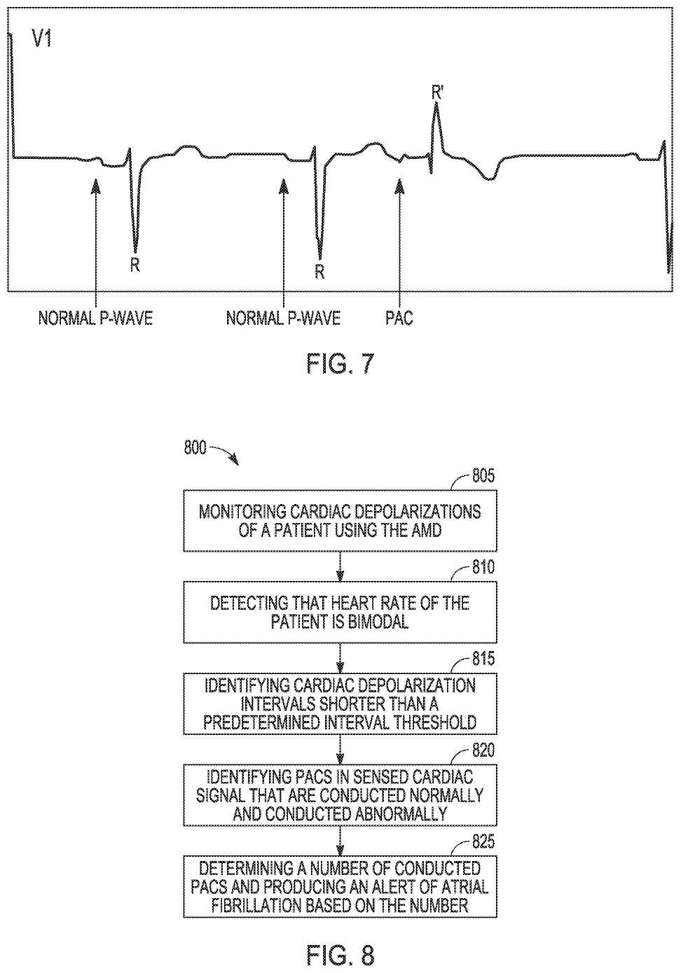

FIG. 7 is an illustration of another example of a strip chart recording of cardiac depolarizations. The example of FIG. 7 shows two normal P-waves resulting in the R-waves labeled (R) and an aberrantly conducted PAC resulting in the R-wave labeled (R′). The R-wave labeled (R′) has a different morphology because the aberrantly conducted PAC may be conducted in an abnormal path to the ventricles causing the ventricles to contract differently than normal. As explained previously herein, knowledge of the PAC burden can be useful to a physician. It may be useful to only include those PACs that are conducted (either normally or aberrantly) in estimating the PAC burden.

FIG. 8 is a flow diagram of an example of operating an AMD 102 (such as the AMD 102 in FIGS. 2 and 3). The control circuit 308 of the AMD 102 performs an algorithm to detect PACs that are conducted to the ventricles. The conducted PACs can be included in a real time estimation of the PAC burden of the patient. The PACs included in the estimation of the conducted PAC burden may be normally conducted (e.g., through the atrioventricular (AV) node 210) or aberrantly conducted (e.g., through a different conduction path than the AV node 210). The PACs that are blocked and not conducted to the ventricles are not included in the estimation of the conducted PAC burden.

At block 805, the cardiac depolarizations of the patient are monitored using the AMD 102. For instance, the control circuit 308 may monitor intervals between R-waves in a cardiac signal sensed by the cardiac signal sensing circuit 304. At block 810, the control circuit 308 detects that the heart rate of the patient is bimodal. One approach to detecting a bimodal heart rate is for the control circuit 308 to produce a histogram of heart rate and detect a bimodal heart rate using the histogram.

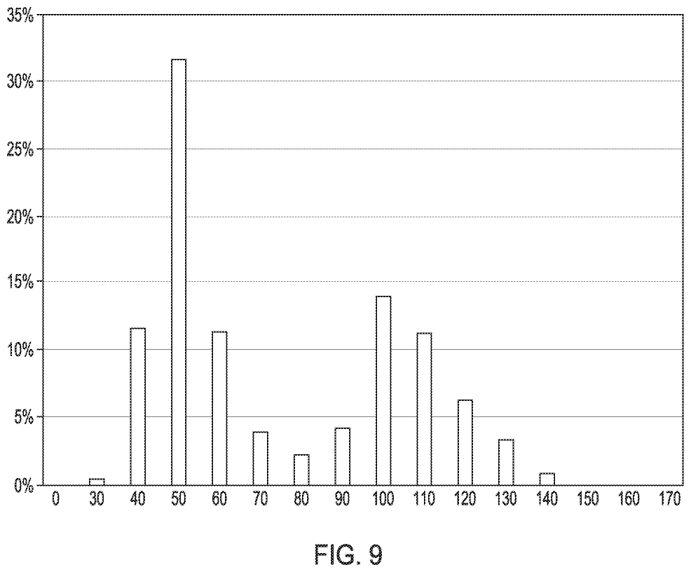

FIG. 9 is an illustration of a histogram in which the monitored heart rate is binned according to beats per minute (bpm). Alternatively, the histogram could use intervals between heart beats (the inverse of heart rate) and the bins could be millivolts. In the example of FIG. 9, the histogram distribution is bimodal with a peak near 50 beats per minute (50 bpm) and a peak near 100 bpm. A histogram for a patient without PACs would have a more normal distribution. The control circuit 308 may detect the bimodal heart rate when identifying the bin having the greatest number of heart beats, identifying a second bin having a large amount of heart beats, and detecting a valley of other bins between the first and second bin. For instance, in the example of FIG. 9, the control circuit 308 would detect a valley of the bin for 80 bpm between the bin for 50 bpm and the bin for 100 bpm, and may set a logic flag for bimodal heart rate when detecting the valley.

Returning to FIG. 8, when the control circuit 308 detects the bimodal heart rate, the control circuit 308 analyzes the sensed cardiac signal to look for fast beats. In some examples, a window of the sensed cardiac signal (e.g., a two-minute window) is stored as the heart rate is monitored and this stored signal is analyzed by the control circuit 308. At block 815, the control circuit 308 identifies cardiac depolarization intervals that are shorter than a predetermined interval threshold as fast beats or short coupling intervals. In some examples, the control circuit 308 uses peak detection to identify R-waves in the sensed cardiac signal and identifies fast R-wave to R-wave intervals as the short coupling intervals. These short coupling intervals are analyzed as potential PAC beats.

At block 820, the control circuit 308 identifies PACs in the short coupling intervals. The control circuit 308 may use a morphology analysis to identify the PACs in the sensed cardiac signal. In some examples, the control circuit 308 compares the morphology of a potential PAC to a PAC template stored in the AMD. In some examples, the control circuit 308 determines a score associated with correlation of the morphology of the sensed cardiac signal to the morphology of a template signal representative of a PAC. An example of a correlation score is a feature correlation coefficient (FCC). The FCC can provide an indication of a degree of similarity between the shape of the sensed electrogram and the shape of the template electrogram signal that represents a PAC. The template may be recorded for a particular subject or may be created based on a patient population. An approach to calculating a correlation score can be found in U.S. Pat. No. 7,904,142, titled “Self-Adjusting ECG Morphological Feature Correlation Threshold.” filed May 16, 2007, which is incorporated herein by reference in its entirety. The arrhythmia detection circuit 620 may detect a PAC when the determined score satisfies a specified PAC detection threshold score. The detection for AF can be adjusted to be more sensitive or less sensitive by adjusting the threshold score.

As shown in the example of FIG. 5, the PACs can be ectopic P-waves that occur before a conducted R-wave. The ectopic P-waves have a different shape than the normal P-waves and occur closer to a previous T-wave than the normal P-waves. The difference in signal morphology can be used to identify ectopic P-waves. In some examples, the control circuit 308 computes a correlation of the potential ectopic P-wave of a short interval to the P-waves belonging to longer intervals. If the shape of the potential ectopic P-wave correlates well with the shape of the other P-waves, it is not identified as an ectopic P-wave. If the potential ectopic P-wave does not correlate well to the other P-waves, it may be identified as an ectopic P-wave. The ectopic P-waves of fast beats that occur before conducted R-waves are identified as conducted PACs and are included in estimate of PAC burden. As shown in the example of FIG. 6, the P-waves that occur without a conducted R-wave are blocked P-waves and the control circuit 308 may not include blocked P-waves when estimating PAC burden.

The identified ectopic P-waves may be PACs that conducted normally or aberrantly. In some examples, the control circuit performs a morphology of the QRS complex of a fast beat to identify aberrantly conducted PACs. As shown in FIG. 7, the morphology of a QRS complex from an aberrantly conducted PAC has a different morphology than the QRS complex from a normally conducted PAC. In some examples, the control circuit 308 computes a correlation score of the QRS complex to the template QRS complex of normal sinus rhythm. If the shape of the QRS complex correlates well with the template QRS, it is identified as a normally conducted PAC. If the shape of the QRS complex does not correlate well with the template QRS, it may be identified as an aberrantly conducted PAC. The control circuit 308 may keep separate counts of normally conducted PACs and aberrantly conducted PACs.

In some examples, the control circuit 308 distinguishes PACs from premature ventricular contractions (PVCs). Detected fast R-wave to R-wave intervals may occur due to PVCs. But fast R-wave intervals caused by PVCs may not include P-waves. The control circuit 308 may identify that a fast R-wave interval is due to a PVC rather than a PAC by the magnitude of sensed signal. In some examples, the control circuit uses P-wave information to distinguish PACs from PVCs. PVCs are not included in the estimate of PAC burden.

Returning to FIG. 8 at block 825, the control circuit 308 keeps a count of the number of identified conducted PACs to estimate conducted PAC burden. The control circuit 308 produces an alert related to PAC burden of the patient when the count of conducted PACs exceeds a predetermined threshold count, or when the count exceeds a threshold count within a predetermined period of time. The PAC burden alert may be a signal transmitted by the AMD 102 to the external device 107. In certain variations, the alert is a flag stored in memory. The external device 107 may receive the alert when it retrieves information stored in the AMD 102. The alert may be displayed by the user interface of the external device, or the external may be a server and the alert is displayed by the user interface of a third device. In some examples, the alert includes a value of the conducted PAC burden. Some examples of the value include a percentage of heart beats with PACs, a number of conducted PACs with a period of time, and the number of normally conducted and the number of aberrantly conducted PACs.

A physician may change the titration of drug therapy, or change another therapy as a result of the alert. In some examples, the control circuit 308 may change a device-based therapy based on the estimate of conducted PAC burden. For instance, the control circuit 308 may initiate delivery of an anti-arrhythmic therapy to treat or avoid AF. In some examples, the therapy circuit 306 provides electrical pacing therapy to the subject. The control circuit 308 may initiate delivery of the electrical pacing therapy according to a first pacing therapy mode and change the pacing therapy mode to avoid AF based on the estimate of conducted PAC burden. In some examples, the external device 107 changes the therapy mode (e.g., by programming the AMD 102) based on the PAC burden alert.

In some examples, the method 800 of FIG. 8 is enabled based on a longer-term monitoring of the patient heart rate. The control circuit 308 may monitor heart rate for a more extended period (e.g., 1-2 weeks) before checking for bimodal distribution of heart rate. If bimodal heart rate is detected over the extended period, the control circuit 308 then enables the PAC burden estimation feature. This can reduce the drain on the AMD 102 energy source (e.g., a battery) from the PAC burden estimation feature. In some examples, the control circuit 308 of the AMD 102 communicates an alert to the external device 107 that the histogram is bimodal without enabling the PAC burden estimation feature. The control circuit 308 may enable the sensing of the depolarization signal and the identifying of the conducted PACs in response to a command received from the external device 107.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. An ambulatory medical device (AMD), the device comprising:
a cardiac signal sensing circuit configured to sense a cardiac signal representative of cardiac activity of a patient when connected to electrodes;
a control circuit configured to:
monitor cardiac depolarizations in the sensed cardiac signal;
detect a bimodal distribution of heart rate of the patient;
identify cardiac depolarization intervals shorter than a predetermined interval threshold;
identify premature atrial contractions (PACs) in the sensed cardiac signal that are conducted normally and conducted aberrantly; and
count a number of conducted PACs and produce an alert related to PAC burden of the patient based on the number.

2. The device of claim 1, wherein the control circuit is configured to identify the PACs by identifying ectopic P-waves before conducted R-waves in the sensed cardiac signal.

3. The device of claim 2, wherein the control circuit is configured to:
compute correlation between QRS complexes in the sensed cardiac signal; and
identify the PACs in the sensed cardiac signal using the correlation.

4. The device of claim 2, wherein the control circuit is configured to analyze ectopic P-wave information and morphology of the sensed cardiac signals to distinguish the PACs from premature ventricular contractions (PVCs).

5. The device of claim 1, wherein the control circuit is configured to analyze morphology of the sensed cardiac signals to identify the PACs that are normally conducted and the PACs that are aberrantly conducted.

6. The device of claim 1, wherein the control circuit is configured to produce a histogram of depolarization intervals of the patient and determine when the histogram is bimodal.

7. The device of claim 1, wherein the control circuit is configured to:
monitor depolarization intervals of the patient;
produce a histogram of the depolarization intervals;
detect when the histogram is bimodal; and
enable the identifying of the PACs during a predetermined timing window of the sensed cardiac signal.

8. The device of claim 7, including:
a communication circuit operatively coupled to the control circuit and configured to communicate information wirelessly to a separate device;
wherein the control circuit is configured to:
monitor the depolarization intervals over multiple days;
communicate an alert to the separate device when determining that the histogram is bimodal; and
receive a command from the separate device to enable the identifying of the PACs in response to a command received from the separate device.

9. The device of claim 1, including:
a communication circuit operatively coupled to the control circuit and configured to communicate information wirelessly to a separate device; and
wherein the control circuit is configured to produce a value of conducted PAC burden and include the value of conducted PAC burden in the alert related to PAC burden of the patient.

10. A method of operating an ambulatory medical device (AMD), the method comprising:
monitoring cardiac depolarizations of a patient in a sensed cardiac signal sensed using the AMD;
detecting a bimodal distribution of heart rate of the patient;
identifying cardiac depolarization intervals shorter than a predetermined interval threshold;
identifying premature atrial contractions (PACs) in the sensed cardiac signal that are conducted normally and conducted aberrantly; and
determining a number of conducted PACs and producing an alert related to PAC burden of the patient based on the number.

11. The method of claim 10, wherein the identifying PACs includes the AMD detecting ectopic P-waves before a conducted R-wave in the sensed cardiac signal.

12. The method of claim 11, including the AMD using ectopic P-wave information and morphology of the sensed cardiac signals to distinguish PACs from premature ventricular contractions (PVCs).

13. The method of claim 10, including the AMD determining when the identified PACs are normally conducted and aberrantly conducted using analysis of a morphology of the sensed depolarization signal.

14. The method of claim 10, wherein the detecting a bimodal distribution of heart rate includes producing a histogram of depolarization intervals of the patient and determining that the histogram is bimodal.

15. The method of claim 10, wherein the detecting a bimodal distribution of heart rate includes:
monitoring the depolarization intervals of the patient;
producing a histogram of the depolarization intervals;
determining that the histogram is bimodal; and
enabling sensing of the depolarization signal in response to the determining that the histogram is bimodal and enabling the identifying of the conducted PACs during a predetermined timing window of the sensed depolarization signal.

16. The method of claim 15, including:
communicating an alert to a separate device when determining that the histogram is bimodal; and
wherein the enabling the sensing of the depolarization signal and the identifying of the conducted PACs includes enabling the sensing of the depolarization signal and the identifying of the conducted PACs in response to a command received from the separate device.

17. The method of claim 10, including the AMD computing a value of PAC burden using the determined number of conducted PACs.

18. A cardiac rhythm management (CRM) system, the system comprising:
an external device including:
a communication circuit configured to communicate information wirelessly with an ambulatory medical device (AMD) when implanted; and
a user interface; and
an implantable AMD including:
a cardiac signal sensing circuit configured to sense a cardiac signal representative of cardiac activity of a patient when connected to electrodes;
a communication circuit configured to wirelessly communicate information to a separate device; and
a control circuit configured to:
monitor cardiac depolarizations in the sensed cardiac signal;
detect a bimodal distribution of heart rate of the patient;
identify cardiac depolarization intervals shorter than a predetermined interval threshold in response to detecting the bimodal heart rate;
identify conducted R-waves in the sensed cardiac signal and detect ectopic P-waves prior to the conducted R-waves;
count a number of conducted ectopic P-waves; and
produce an alert related to PAC burden of the patient based on the number of conducted ectopic P-waves and communicate the alert to the external device.

19. The system of claim 18, wherein the external device is configured to:
retrieve information stored in memory of the AMD;
receive the alert related to PAC burden in the retrieved information; and
display the alert related to PAC burden to a user of the external device.

20. The system of claim 18,
wherein the AMD includes a therapy circuit to provide electrical pacing therapy to the patient when coupled to the electrodes; and
wherein the external device is an AMD programmer configured to communicate a command to the AMD to change a therapy mode in response to receiving the alert related to PAC burden.
